(19) United States
(12) Patent Application Publication
(10) Pub. No.: US 2018/0263574 A1
(43) Pub. Date: Sep. 20, 2018

(54) System for the capture and combined display of video and analog signals coming from electromedical instruments and equipment

(71) Applicant: SPARKBIO S.R.L., I-40068 San Lazzaro di Savena (IT)

(72) Inventor: Sebastiano ZANNOLI, Bologna (IT)

(21) Appl. No.: 15/534,660

(22) PCT Filed: Dec. 10, 2015
(86) PCT No.: PCT/IB2015/059516

§ 371 Date: Jun. 9, 2017

(30) Foreign Application Priority Data
Dec. 10, 2014 (IT) BO2014A000691

(51) Int. Cl. A61B 5/00; H04N 5/445; H04N 7/10; H04N 5/44; H04N 7/08; H04N 5/06; A61B 6/00; A61B 8/00; A61B 8/08; A61B 90/00

(52) U.S. Cl. CPC A61B 5/7425; G06K 9/62; H04N 5/44504; H04N 7/104; H04N 5/4403; H04N 7/08; H04N 5/06; A61B 5/7289; A61B 6/463; A61B 6/5288; A61B 8/463; A61B 8/5284; A61B 90/361; G06K 2209/01; G06K 2209/05; H04N 5/44591

(57) ABSTRACT

A system is proposed for the acquisition and the combined displaying of video output signals, for example in the VGA standard, coming from a set of various instruments and apparatuses (2a, 2b, 2c, . . . 2n), each of which reproduces at least a signal representing the evolution of a physiological parameter, a diagnostic image of a part of the body of a person, or both. The system collects a video signal in the section connecting each of the instruments and apparatuses (2a, 2b, 2c, . . . 2n) with a monitor (3a, 3b, 3c, . . . 3n). The video signals are carried to a central unit (1) for collection and processing of the signals and then presented in a single image on a central monitor (7).

### TECHNICAL FIELD

The present invention relates to the technical field concerning electromedical equipment for monitoring physiological parameters of patients undergoing surgery or interventional procedures. In particular, the invention concerns a system for the collection and display of synchronized signals coming from different instruments and equipment, which are used by different operators for therapeutic or monitoring purposes.

The present invention will be described and claimed in the following preferably with reference to the medical, surgical and interventional fields, for sake of simplicity and clarity. However, it shall be understood that this choice has exemplary purposes only and does not imply any environmental or functional limitation in relation to its application.

### BACKGROUND ART

As it is known, many devices having different functions are present in the operating room in the course of surgery and/or interventional procedures. For example, a surgical robot, an angiographic arc for radiological investigation, a polygraph or multiparameter monitor that allows to display different signals corresponding, for example, to an electrocardiogram or blood pressure, or an echo doppler instrument, are commonly used. In addition, device programmers can be used to define the functionality of devices applied to the patient during the procedure or after the surgery, such as a pacemaker or a pump for external ventricular assist.

Each of these devices produces its own set of information data, that refer to one or more parameters related to the state of the patient, and in particular to the trend of the physiological ones, which must be kept under constant control during the intervention. These information data, used to control certain functions, are usually represented graphically, transformed into representative numerical values, then displayed in the form of constantly updated images, and stored in storage media present within each single apparatus, or in another way, by means of devices connected thereto.

For standardization purpose, the information data are transformed into suitable formats, such as those known with the abbreviations “DICOM” or “HL7”. These formats allow an easy storage of the information data in large storage media, which are also shared with all devices. This procedure allows to form many separate files collected in a single large memory device. However, this procedure does not allow any verification of the value or evolution of a parameter at a time when, according to the information obtained from another apparatus, a specific event occurs.

### TECHNICAL PROBLEM

In more explicit terms and by way of example, checking how the situation evolves would be particularly useful for the surgeon or who is in charge of monitoring the patient's condition, as detected, for example, from the ultrasound image and at the same time from the electrocardiogram trend. Again by way of example, if the electrocardiogram shows a particular pathological event that affects the heart, verifying the condition of patency of the blood perfusion system to the heart at that precise moment, by means of the archive, could be useful to determine the cause of the event through the evaluation, at a later time, of the image obtained from the angiographic X-ray system.

To carry out this check with the current systems, the electrocardiogram printout should be first retrieved and the exact temporal position at which the pathological event occurred should be identified. Then the time corresponding to the position on the electrocardiogram must be identified in the sequence of angiographic frames, with the assumption that the timing of the angiographic report corresponds perfectly to that of the electrocardiogram graph. But this condition does not often occur in actual operations.

This example can be extended to any other physiological parameter or pathological or non-pathological event, always finding the same problem of retrieval and synchronization of the various signals with the necessary precision.

In the operational context of an operating room as well as in other areas where complex diagnostic investigations are performed, there is the need to control the various parameters and significant physiological data of the patient, which are taken from different instruments, at the same time, as they become available. This would allow to have a view and an evaluation of these parameters and data simultaneously on a single display, and operations could possibly be carried out on these parameters and data to highlight particular situations while always keeping records of the moments in which they occurred.

There are known systems, also in the medical-surgery field, that allow the acquisition of signals coming from the main and auxiliary analog outputs of one or more instruments, such as from electro-medical equipment, gathering them in a specialized computer, in order to store and process the same signals and provide useful information about the same.

The publication “DESIGN OF A SOFTWARE MODULE FOR SIMULTANEOUS ACQUISITION OF SIGNALS AND IMAGES IN NEUROPHYSIOLOGICAL FIELD” of Dec. 20, 2012, as the graduation thesis by SIMONA ANZIVINO, discloses a device of this type, known under the trade name “LIGHT”. This apparatus comprises essentially a plurality of analog-digital converters, which are intended to acquire information data related to the amplitude of the output signals from dedicated signal lines. The signal lines originate from different instruments used for tests in the field of neurophysiology. A computer, provided with an adequate number of digital inputs and a dedicated analysis software, performs clinical and statistical analysis of each acquired physiological parameter and correlation analysis of the different physiological parameters, based on diagnostic evaluations to be carried out on a patient.

The above described apparatus, as regards the data displaying, works substantially in the same way as a standard polygraph, displaying on a monitor at the same time the traces acquired in graphical form and printing the reports of analyses carried out on the acquired signals. Possible footages acquired with the environmental cameras are displayed on a separate monitor. For this purpose, it should be noted that a polygraph is an instrument designed to detect, acquire and display simultaneously neurophysiological and cardiovascular signals, such as blood pressure, ECG (electrocardiogram), EEG (electroencephalogram) and breathing activity.

On the other hand, the US Patent Application No. US2008/058963 (Garibaldi et al.) addresses a different technical problem and describes an interactive standardized control device for controlling the operation of different electromedical equipment. For this purpose, the device includes a monitor, which can be controlled to display what a particular apparatus shows on its screen at that moment, whereas what is displayed on the other equipment is substantially reduced to icons situated in inactive areas of the monitor.

However, the above described equipment does not satisfy the needs indicated before, since the functions involved are dedicated to processing and storing the quantitative characteristics of the single acquired parameters (Anzivino) or improving the control of the operation of each single apparatus (Garibaldi).

### OBJECTS OF THE INVENTION

The object of the present invention is to propose a system for acquiring and combined, synchronized displaying of video signals coming from electromedical equipment, camera footage and possibly images formed from analog signals, which allows the operators in the operating room to see more events and physiological parameters at the same time, so as to obtain and watch the evolution of the correlated physiological parameters in the same moment in which a given event occurs, and allow the identification of the possible cause of that event with improved certainty.

Another object of the invention is to propose the above mentioned system which is able to make possible an accurate reconstruction of the events in the operating room, making it continuously available and sharing it with other involved subjects.

A further object of the present invention is to implement the above mentioned system in a simple but reliable way, without the necessity to replace the equipment which is already present and working in the operating room and without the necessity to apply further detectors, probes, etc. to the patient.

The proposed system should not interfere in any way with the normal activity of the medical team while carrying out the operation, it should not give additional information apart from the data already obtained from the parameters under control, but it must simply make it possible to display all the data already obtained synchronized together, in real time or delayed.

### SUMMARY OF THE INVENTION

These and other objects are obtained by the system proposed by the present invention, carried out by a system according to a first aspect of the present invention. Such a system is provided for the capture and display of output signals coming from a set of various electromedical instruments and apparatuses, each comprising display means, for displaying the operations carried out by said electromedical instruments and apparatuses, said operations including a trend of at least one physiological parameter of a patient, or a diagnostic image or video clip taken during a diagnostic check-up or an operation, said system including:

video signal shunt devices, installed in corresponding sections connecting the output signals of each of said instruments and apparatuses with corresponding related display means, said video shunt devices being fit to shunt said video signals in order to make the corresponding video outputs available in a corresponding standard as used by said instruments or apparatuses;

a plurality of connections, respectively arranged between each video signal shunt device, and a central unit for collection and processing of signals;

video capture means provided in said central unit, connected to said plurality of connections, for capturing video outputs coming from said video signal shunt devices, for making said video outputs available for storing in said central unit for combined display;

synchronization means for synchronizing various images or sequences of images coming from said video capture means, and defining time reference signals for said images;

a processing computer program operating in said central unit, for processing said various images or sequences of images to obtain corresponding combined images or sequences of images, in which two or more of said images or sequences of images coming from the set of various electromedical instruments and apparatuses, are composed in a single combined image or sequence of images combined in synchrony; and

further display means, connected to said central unit, for displaying said combined images or sequences of images.

According to a second aspect of the present invention, a method is provided for presentation of sensed data, comprising:

receiving sensed signals from separate electromedical instruments, indicative of correspondingly separate parameters of a subject undergoing corresponding physiological changes during an operation, and

processing the received signals in a central unit to synchronize the received signals and to present a video signal to a display for displaying corresponding images or sequences of images of the separate parameters combined for real time display altogether on a single display as a combined image for real time comparison of the corresponding physiological changes.

According to a third aspect of the present invention, a nontransitory storage medium is provided having a computer program stored thereon, the computer program executable by a processor to execute the steps of the method according to the second aspect of the present invention.

Further characteristics and versions of the system are described below.

### BRIEF DESCRIPTION OF THE DRAWINGS

The characteristics of the invention which do not appear from what has been said before, are pointed out in the following description, with reference to the enclosed tables of drawings, in which:

FIG. 1 is a simplified block diagram of the system proposed by the present invention;

FIG. 2 is a possible graph which is made available by the system of FIG. 1.

FIG. 3 illustrates a combined image displayed on a monitor, consisting of a scenario that comprises the composition of the video output of an X-ray scopy apparatus, of a pacemaker programmer and the shooting of a environmental camera situated in the operating room.

### DESCRIPTION OF PREFERRED EMBODIMENTS OF THE INVENTION

With reference to the above mentioned Figures, a system has been illustrated aimed at collecting and displaying synchronously the output signals coming from a group of different instruments and apparatuses 2a, 2b, 2c, . . . 2n, normally used in the operating room during a surgery operation or an interventional procedure.

As already said in the introductory note, the present treatment refers also to the specific environment of an operating room, but this must not be considered a limit, as the invention can be validly applied also in other similar fields.

Again by way of not limiting example, it is specified that the set of different instruments and equipment 2a, 2b, 2c, . . . 2n may comprise, among others, a surgical robot, an angiographic arc and/or a polygraph or a multiparameter monitor, and/or thoracic strain-gage, and/or an echo doppler console, and/or a series of programmer appliance for medical devices and all the monitoring instrumentation and anesthesia and breathing support. Obviously, the list is not exhaustive and in any case, there can be only a part of the listed instruments and apparatuses.

Each of the instruments and each of the apparatuses 2a, 2b, 2c, . . . 2n includes display means 3a, 3b, 3c, . . . 3n for displaying an image representing the evolution of a physiological parameter of a person, or of an image of a part of the body or of an apparatus of the same person, taken during an operation. It will be appreciated that such images are generally taken as video clip or continuous film, with a frame rate varying from some frames per second to some tens of frames per second. For the purposes of the present invention, such a video will be referred to as “image”, to indicate a single frame, or “sequences of images”, to indicate the video or its parts.

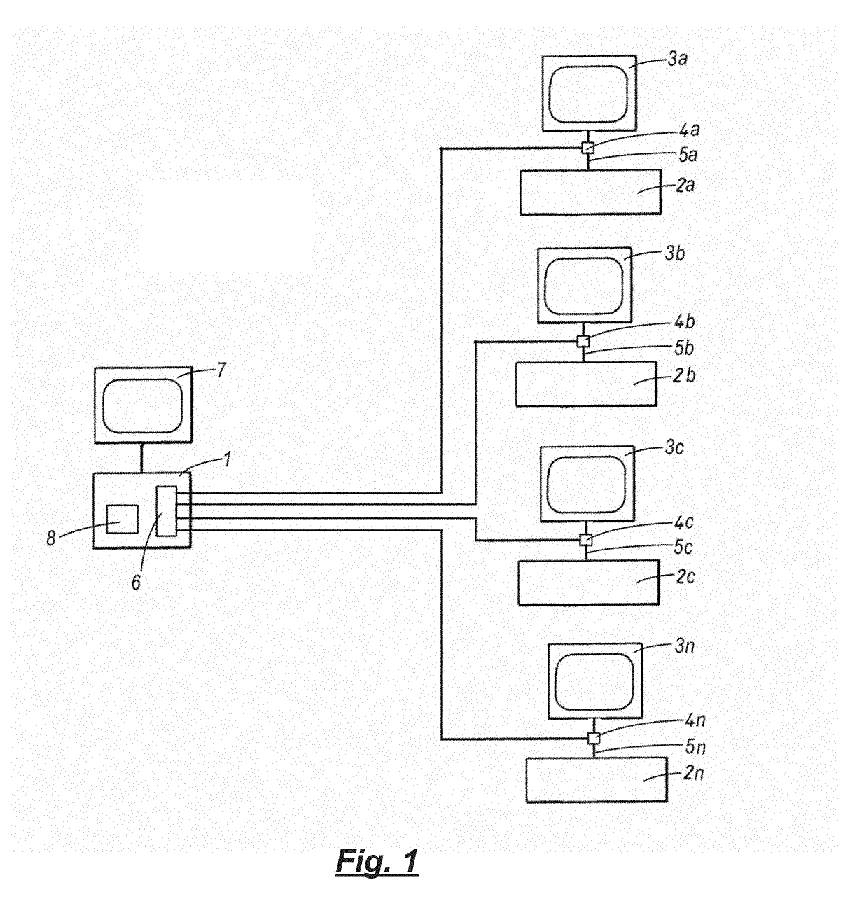

In addition, in particular, the display means 3a, 3b, 3c, . . . 3n include a normal monitor, for example a VGA monitor or another video format monitor (FIG. 1), such as a composite video, DVI, HDMI, etc.

VGA monitors or another video format monitor are connected to the video outputs of the instruments and apparatuses 2a, 2b, 2c, . . . 2n, by suitable cables.

It will be appreciated that in the equipment or instruments 2a, 2b, 2c, . . . 2n which can be used for the system according to the invention, the video monitors can be also integrated, as long as they are provided with a direct video output line, such as a VGA or another industrial video standard (as already mentioned, for example a composite video, a DVI, HDMI standard, etc.). In the following reference will be made to the VGA standard, but only by way of example.

In order to avoid any conditioning of the standard operation of the room, according to the invention, in the connecting section 5a, 5b, 5c, . . . 5n of each of said instruments and each of said apparatuses 2a, 2b, 2c, . . . 2n, with the related display means 3a, 3b, 3c, . . . 3n, there are installed as many analog video signal shunt devices 4a, 4b, 4c, . . . 4n, in particular VGA splitters or another video format splitters, of a commonly commercially available type.

One of the outputs of each splitter 4a, 4b, 4c, . . . 4n is again connected to the video monitor of the related instrument or related apparatus, in such a way as to resume previous normal operation.

A plurality of shunt connections 5a, 5b, 5c, . . . 5n connect the other outputs of the splitters of the analog video signal 4a, 4b, 4c, . . . 4n to a central unit 1 for collection and processing of signals. The central unit 1 comprises means, devices and memories for running processing programs suitable for the tasks it has to carry out, as it will become evident later on.

In particular, the shunt connections 5a, 5b, 5c, . . . 5n lead to video capture means 6, formed by one or more capture cards, capable of acquiring the signals in the format produced by the equipment 2a, 2b, 2c, . . . 2n, for example, the VGA format, and of storing them in suitable memory areas provided in the central unit 1. Also these capture cards, commonly available commercially and provided with a number of capture channels, at least as many as the connections 5a, 5b, 5c, . . . 5n, are likewise installed in the central unit 1. In this way, each of the received split signals are converted in sequences of digital images, for each of which a temporal reference is defined. Each sequence of images related to each capture channel is stored in corresponding video files, which preferably include associated information regarding the acquisition time.

The so obtained digital images are combined in a single image, by means of a suitable processing computer program, resident and operating in the central unit 1, to obtain corresponding composite images, in which two or more such digital images are suitably placed in the combined image, depending on the displaying scenarios defined by an operator. The composite image is then reproduced on the displaying means 7, which are composed by a monitor connected with, or integrated with the above mentioned central unit 1.

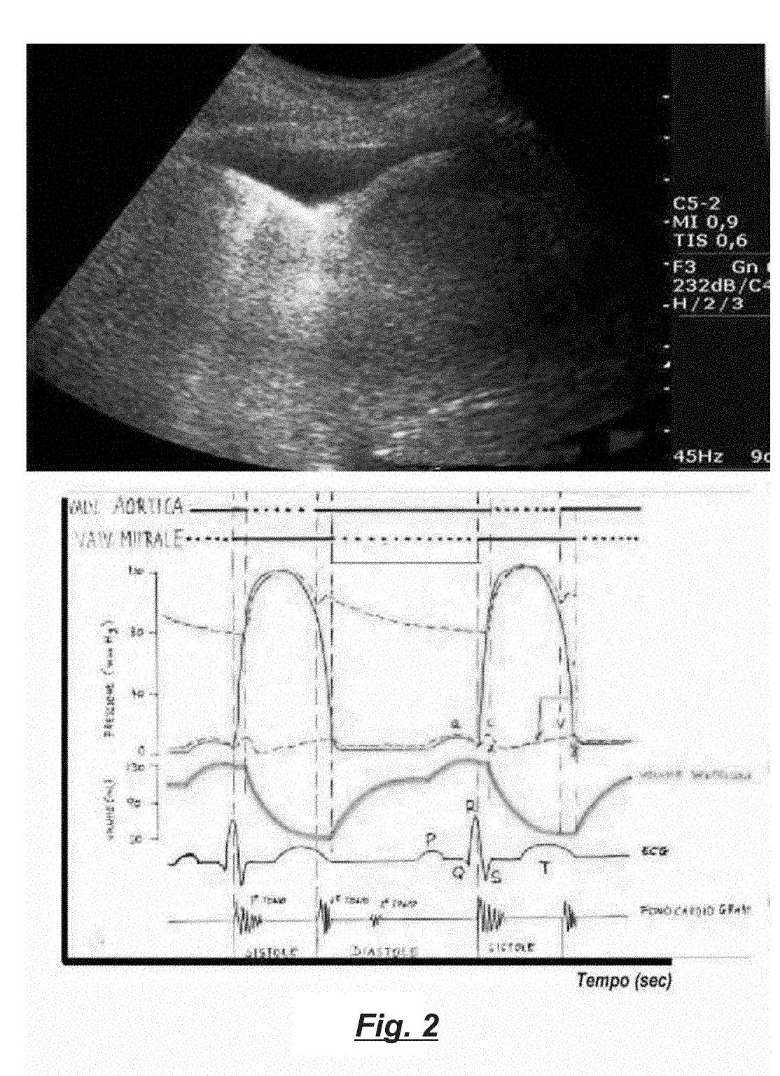

The above described operation is repeated continuously for each photogram acquired at the same moment by different capture channels, to obtain at the output on the monitor 7 a continuous real-time video clip, which shows the footages made by the apparatuses or instruments 2a, 2b, 2c, . . . 2n selected in the presently defined scenario (see FIG. 2). A procedure for modification of the scenario is provided to allow the operator to change the displayed scenario in every moment and to replace it with other scenarios possibly defined before or at once.

In the case in which the different sources of video signals operate with different reproduction rates of the screens (“frame-rate”), the software procedure for acquiring the signals with a slower frame-rate advantageously copies a frame of the related video in such a way as to obtain a constant number of frames for time unit for all the sources acquired and reproduced on the monitor 7.

For each scenario displayed on the monitor 7 there is the possibility, by means of a selecting procedure that is a part of the processing computer program, of selecting a preferred image from those appearing in the scenario, and showing it enlarged with respect to the others, since at this moment it is more significant for the person who carries out the operation. Moreover, it is possible for the operator to order the substitution of such a preferred image inside the scenario with another image of the same scenario.

In another case, it is possible to re-examine the images in the post-processing mode one by one or all together.

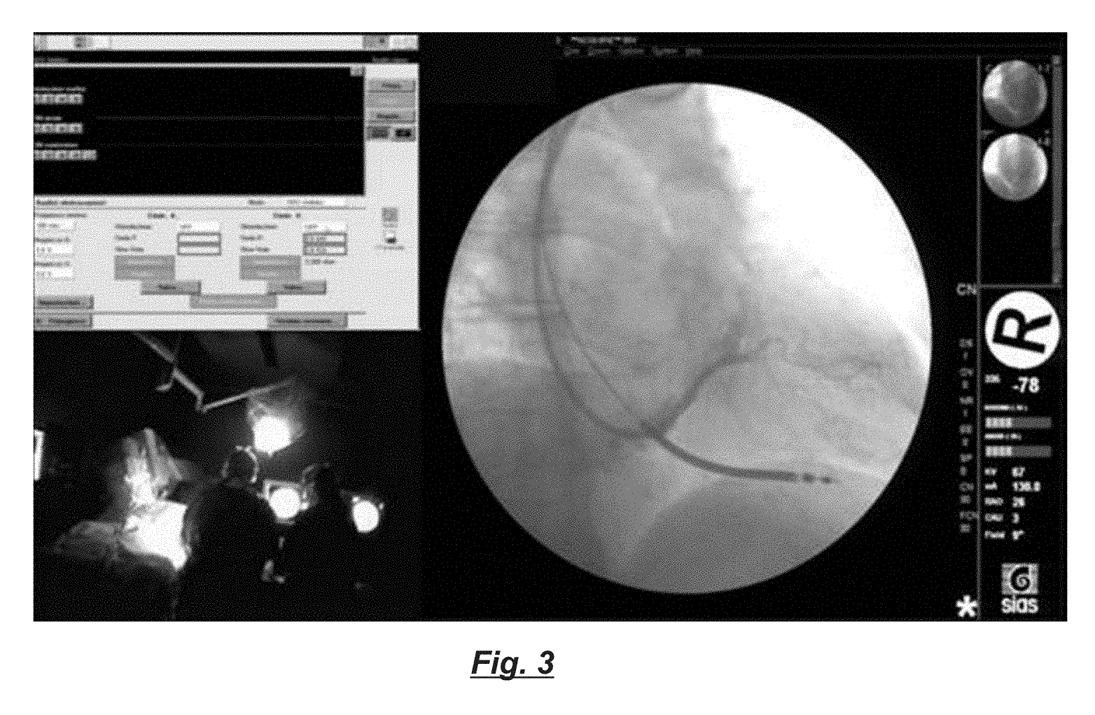

By way of example, FIG. 3 illustrates a combined image displayed on the monitor 7, consisting of a scenario that comprises the composition of the video output of an X-ray scopy apparatus, of a pacemaker programmer and the shooting of a environmental camera situated in the operating room. In this case, the preferred image of the scenario is that supplied by the X-ray scopy apparatus.

Therefore, the operators who are carrying out a surgery can control by only one monitor the trend of the physiological parameters significant for the occurrence of certain events that can be detected by other monitored parameters, and to control all this by displaying at the same time the image taken by a diagnostic or surgery instrument used in that moment on the patient. For example, in a moment in which a pressure drop occurs, the behavior of the walls of the heart can be checked in the ultrasound image or other physiological parameters connected to a possible variation of the pressure can be detected.

The possibilities of processing and displaying the combinations of various images and footages are extremely numerous, since one can highlight one of them, enlarging it or making it more pronounced with respect to the others, which are to be kept in the background. All these possibilities will become evident and within the reach of those skilled in the art.

In order to keep records of the acquired and displayed images, in the central unit 1 there are provided storage means 8, independent for each digital image or sequence of images, received from each instrument and/or each apparatus 2a, 2b, 2c, . . . 2n. This allows an operator, if he considers it useful, to invoke the images or the sequences in a second step, and to analyze them, one by one or in any combination thereof.

In order to make the system proposed by the present invention more functional, the central unit 1 comprises a processing and conversion computer program, resident in a suitable memory in the central unit 1 and operating by means of a suitable electronic board for analog-to-digital conversion, for the sampling of one or more analog signals. This program is adapted to acquire and convert to the digital format the non video outputs of the instruments 2a, 2b, 2c, . . . 2n, which supply analog signals, whose amplitude is correlated with the value acquired in a moment by the parameter or parameters they are monitoring. The acquisition and conversion program is also aimed at generating corresponding images or sequences of images of the value of the above mentioned parameter, or of the above mentioned parameters, adapted to be introduced in the combined images described above.

For example, the polygraph is provided with an output, which emits a voltage signal corresponding to the electrocardiogram, represented at the same time on the related monitor, as it can be well seen in FIG. 2, which illustrates an exemplifying display of the monitor 7.

Advantageously, timing of the sampling of the analog signals can be carried out using the synchronizing signal generated by the sampling board of one of the available video signals, coming from the different apparatuses, as a synchronizing signal for the analog-to-digital conversion board. In this way, each “frame” of the acquired video has its corresponding single sampling of the analog signals. Using such synchronizing signal as reference for the sampling of analog signals always ensures perfect synchrony between the video and analog channels.

This allows to draw, together with the superimposed digital images, a corresponding diagram, which reports the real analog values of the monitored physiological parameters, allowing to assign corresponding values to the images being shown, which usually allow a more qualitative evaluation.

Furthermore, still in order to give the system improved functionality and information capacity, the processing computer program comprises a procedure for character recognition, aimed at detecting the graphical representation of numerical values representing a physiological parameter, which are normally reproduced inside the relative digital images in predetermined positions. In practice, this is a common OCR program, that is an alphanumeric character recognition from the graphical representation in a matrix of pixels.

The object is to derive a precise value used individually or in combination with other values, obtained in the same way from other images acquired at the same time or with the value of the sampled analog signals, to allow to carry out further diagnostic evaluations, also with reference to the superimposed representation of the images related to the various monitored physiological parameters.

For example, if in a particular moment the blood pressure assumes an anomalous value, reference can be made to the ultrasound image of the same moment so as to derive, by means of the OCR program, the precise value corresponding to the contractility of the wall of the heart, from the value reported graphically in a given position, usually fixed, and consequently, it is possible to identify a relation between the detected contractility of the wall of the heart and the anomalous value of the pressure.

The processing computer program that manages the functions of the system in the central unit 1 comprises also advantageously, a procedure for insertion of time markers in correspondence to the images related to significant events of an operating session. These markers can be inserted by an operator by means of the normal input interfaces present in the central unit 1, for example a mouse or a keyboard, either at the moment in which the significant event is occurring or in a subsequent post-acquisition phase. A comment can be introduced in correspondence to each marker, or measurements of the captured analog signals can be introduced at that moment or immediately before or immediately after. The markers and the related notes are also stored in the mass memory of the central unit 1.

The processing computer program comprises also a procedure for generating video files in a standard format (for example .AVI format), from the sequences of combined images, for the scenario displayed on the monitor 7 in that moment and for the whole duration of the operation.

In addition, the processing computer program can comprise also an extraction procedure, designed to be enabled by a particular instruction issued by an operator. As a result of such instruction, the procedure extracts from the sequence of combined images being acquired and stored a video file, whose duration is comprised between a pre-defined previous time period of acquisition and a pre-defined subsequent time period of acquisition with respect to the moment in which the instruction has been received. This video can comprise, for example, the 20 seconds before and the 20 seconds after the instruction, to fix a particular passage of the operation being carried out and make it available later on.

Consequently, the generated video files can be made available for exportation and for subsequent displaying on any informatic or computer channel, as a document of the real evolution of the operation and what was desired to be displayed by the person who carries out the operation in order to control the instrumentation of the operating room.

A function of the system according to the invention consists also in sharing the video clip displayed on the monitor 7, with a remote interlocutor, in real-time, so as to make the events and significant parameters of the operation available also to the interlocutor. For this purpose, the central unit 1 can be provided with particular commands to activate a mode for the connection with one or more pre-defined interlocutors. This can be carried out, for example, by means of a software procedure that receives the command for a given remote interlocutor, sends, via email, to the address of such interlocutor an invitation containing an address for the connection to the video being made and enables his/her access after having validated the respective credentials.

The diagnostic evaluations, or even only suggestions related thereto, can be formulated also in an automatic way by a suitable procedure which is a part of the processing computer program. This procedure is intended to analyze and compare, possibly also with models contained in a suitable database present locally or accessible via a network, the trend of the representative values corresponding to the physiological parameters and digital images detected to supply in real time diagnostic hints to the person who carries out the operation.

More in general, it is possible for the program or the health professional to combine one or more of the signals, of the images or measurements obtained therefrom or from the analog signals, to obtain more complete and accurate diagnostic indication, moment after moment, useful in different situations.

It will be appreciated that the objects mentioned in the introductory note have been fully obtained. The described system gives a series of information data either from the analog data or from images produced by the different present instruments and equipment, in order to process, combine, reproduce and analyze them in such a way as to perform overall evaluations which account for more events and measurements in real time.

Therefore, identification and localization of the causes of given pathologic events present an accuracy and adherence to reality that were not possible to obtain with a separate analysis of the various parameters in different moments, as carried out up till now.

It is understood that the described system can be used also in other areas and in other fields, applying suitable variations that in any case are included in the scope of the invention in question, without departing from the spirit of the invention.

It is also understood that different embodiments or variants of the present system remain within the protection scope given to the present invention as described above and defined in the following claims.

1. A system for the capture and display of output signals coming from a set of various electromedical instruments and apparatuses, each comprising display means, for displaying the operations carried out by said electromedical instruments and apparatuses, and operations including a trend of at least one physiological parameter of a patient, or a diagnostic image or video clip taken during a diagnostic check-up or an operation, said system including:
video signal shunt devices, installed in corresponding sections connecting the output signals of each of said instruments and apparatuses with corresponding related display means, said video shunt devices being fit to shunt said video signals in order to make the corresponding video outputs available in a corresponding standard as used by said instruments or apparatuses;
a plurality of connections, respectively arranged between each video signal shunt device, and a central unit for collection and processing of signals;
video capture mans provided in said central unit, connected to said plurality of connections, for capturing video outputs coming from said video signal shunt devices for making said video outputs available for storing in said central unit for combined display;
synchronization means for synchronizing various images or sequences of images coming from said video capture means, and defining time reference signals for said images;
a processing computer program operating in said central unit, for processing said various images or sequences of images to obtain corresponding combined images or sequences of images, in which two or more of said images or sequences of images coming from the set of various electromedical instruments and apparatuses, are composed in a single combined image or sequence of images combined in synchrony; and
further display means, connected to said central unit, for displaying said combined images or sequences of images.

2. A system as claimed in claim 1, wherein one or more of said instruments and apparatuses are provided with analog signal outputs, the width of said signals being correlated with the evolution of corresponding physiological parameters being monitored, and in which said central unit comprises analog sampling and conversion program means for detecting said analog signal outputs and for generating corresponding digital images or sequences of images fit to be inserted into said combined images or sequences of images.

3. A system as claimed in claim 2, wherein said processing computer program includes a procedure for character recognition, fit to acquire graphical representations of numerical values representing a physiological parameter, printed within one of said digital images of sequences of images, in order to derive a precise value to be used individually or in combination with other values.

4. A system as claimed in claim 3, wherein said processing program comprises a diagnostic analysis procedure, fit to perform a diagnostic evaluation based on representative values corresponding to the physiological parameters and the digital images detected and considered in a partial or total combination.

5. A system as claimed in claim 1, wherein said display means, are each constituted by a VGA monitor or other video format monitor, connected to a corresponding video output of a respective instrument, a respective apparatus, or both, and said analog video signal shunt devices, each include a VGA video signal splitter, or a splitter working with another video format.

6. A system as claimed in claim 1, wherein one or more of said display means comprise an auxiliary video output line, and wherein corresponding one or more video signal shunt devices leading to the aforementioned central unit (1) are formed by corresponding video cables.

7. A system as claimed in claim 1, wherein said processing computer program is set in continuous operation to generate and store sequences of said combined images with predetermined rate, with time references being assigned to said sequences in order to define an exact acquisition time.

8. A system as claimed in claim 7, wherein said processing program further includes a procedure for the generation of standard format video files adapted to be exported and displayed in contexts external to said system.

9. A system as claimed in claim 1, wherein said processing means further include a procedure for insertion of time markers at the occurrences of significant events of an operating session, said time markers being defined at the time of acquisition or in a subsequent processing step.

10. A system as claimed in claim 1, wherein said combined image, or sequence of combined images, comprises a subset of said images or sequences of images, a procedure for modification of a scenario being provided by said processing computer program in order to allow an operator to define and modify said subset of images or sequences of images.

11. A system as claimed in claim 10, wherein said processing computer program comprises a procedure for selecting, within said subset, a preferred image or sequence of images to be displayed with a bigger size with respect to other images or sequences of images in said combined image or sequence of images in order to enhance its visibility, or to substitute said preferred image or sequence of images with another image or sequence of a same scenario.

12. A system as claimed in claim 1, wherein said processing computer program comprises an extraction procedure, that detects a particular instruction issued by an operator and, consequently, to extract from said sequence of combined images being acquired, a video file comprising a pre-defined previous time period of acquisition and a pre-defined subsequent time period of acquisition of said sequence of combined images, with respect to the time of detection of said instruction.

13. A system as claimed in claim 1, wherein said processing computer program includes also a real-time remote sharing procedure, fit to allow said combined image or sequence of combined images to be shared with third parties.

14. A method for presentation of sensed data, comprising:
receiving sensed signals, each indicative of a separate physiological parameter of a subject undergoing physiological changes during an operation, the sensed signals received from corresponding separate instruments, and
processing the received signals in a central unit to synchronize the received sensed signals and to present a video signal to a display for displaying corresponding images or sequences of images representative of the separate parameters combined for real time display altogether on a single display as a combined set of images for real time comparison of physiological parameters of the subject undergoing the physiological changes.

15. A nontransitory storage medium having a computer program stored thereon, the computer program executable by a processor to execute the steps of the method of claim 14.
